(19) United States
(12) Plant Patent Application Publication
(10) Pub. No.: US 2002/0144325 P1
(43) Pub. Date: Oct. 3, 2002

(54) VERBENA PLANT NAMED 'BLANCENA'

(76) Inventor: Henricus G.W. Stemkens, Hoorn (NL)

Correspondence Address: Larry W. Stults, Syngenta, Patent Deparatment, P.O. Box 12257, Research Triangle Park, NC, 27709-2257, US

(21) Appl. No.: 09/819,228

(22) Filed: Mar. 28, 2001

(51) Int. Cl. A01H 5/00

(52) U.S. Cl. PLT/308

(57) ABSTRACT

A new Verbena plant particularly distinguished by its very large white flower, early flowering, and a spreading habit.

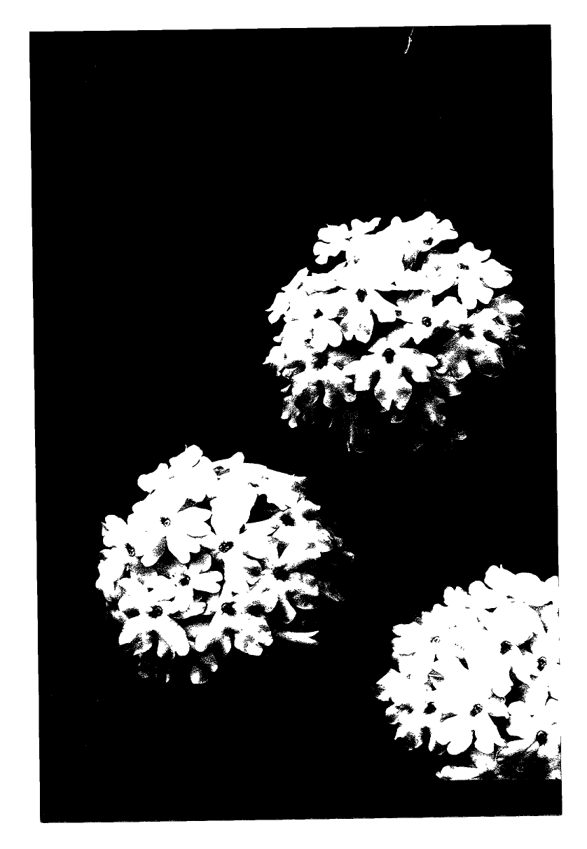

### BACKGROUND OF THE NEW PLANT

The present invention comprises a new distinct cultivar of Verbena, botanically known as Verbena*hybrida. The new cultivar is propagated from cuttings resulting from the cross of Z736 and W688. Z736 is a white flowering Verbena having a spreading habit. Z736 is not commercially available and is not known by any synonyms. W688 is a white flowering Verbena having a spreading habit. W688 is not commercially available and is not known by any synonyms. Neither Z736 or W688 has been patented. As a result of this cross the present cultivar was created in 1997 in Enkhuizen, Netherlands and has been repeatedly asexually reproduced by cuttings in Enkhuizen, Netherlands and Sarrians, France over a three year period. It has been found to retain its distinctive characteristics through successive propagations, and this novelty appears to be firmly fixed.

This new Verbena plant is an annual in most climatical zones in the US, only in zones 9 and 10 it is a perennial plant.

### DESCRIPTION OF THE DRAWING

This new Verbena plant is illustrated by the accompanying photographic drawing which shows blooms, buds and foliage of the plant in full colour, the colour shown being as true as can be reasonably obtained by conventional photographic procedures.

### DESCRIPTION OF THE NEW CULTIVAR

The following detailed descriptions set forth the distinctive characteristics of this new Verbena. The data which defines these characteristics were collected from asexual reproductions carried out in Enkhuizen, Netherlands. The plant history was taken on 14 weeks old plants, blossomed under natural light in a greenhouse. Colour readings were taken in the greenhouse under ambient light. Colour references are primarily to the RHS Colour Chart of The Royal Horticultural Society of London.

The plant:

Classification.—Botanical: Verbena*hybrida.

Parentage.—Female parent: A seedling named Z736 is one of our seedlings from our Z-generation of plants bred in 1996. Pollen parent: A seedling named W688 is one of our seedlings from our W-generation of plants bred in 1992.

Growth habit.—Spreading.

Plant height.—20-25 cm.

Spreading area of plant.—80-120 cm.

Growth rate.—Hanging and vigorous.

Strength.—Good

Branching Character.—Good branching.

Blooming period.—From April till November.

The stem:

Diameter.—2-3 mm.

Shape.—Tetragonal.

Anthocyan pigmentation.—Absent.

Length of internode.—30-70 mm, depending on the light where the plant is propagated.

Pubescence.—Pubescence is present.

The foliage:

Phyllotaxis.—Opposite.

Shape of blade.—Broadly ovate.

Texture.—Simple lobed.

Length.—35-50 mm.

Width.—30-40 mm.

Depth of incision.—2-3 mm, toothed.

Colour.—Upper side: Medium green 141B Lower side: Light green 139C.

Pubescence.—Some pubescence is present.

Length of leaf stem.—5-10 mm.

The bud:

Peduncle length.—20-45 mm, depending on season.

Size.—Diameter.—2 mm. Length: 8-12 mm.

Shape.—Long.

Colour.—Light green 139C.

Sepals.—Colour: Green 137D Form: Standing up.

The flower:

Direction.—Ascending.

Outward curvation of petal.—Medium curved.

Diameter.—19-23 mm.

Height.—18-22 mm.

Borne.—In a cluster.

Form.—Horizontal symmetric.

Cluster.—Umbel.

Colour.—White 155C.

Eye.—A very small green-white eye is present. Typically three out of five petals exhibit this green-white coloration.

Overlapping of petals.—Separate.

No. of petals.—5.

Shape of the petals.—Gamopetalos, grown together at the base of the petal. Each petal is heart shaped at the top.

Size of the petal.—Length.—7-10 mm Width: 8-9 mm.

Spike.—Length: 20-45 mm Diameter: 40-60 mm.

Calyx.—Length of 10-12 mm.

Anthocyan pigmentation of calyx limb.—Absent.

No. of flowers per spike.—25-40.

Fragrance.—No fragrance.

Reproductive organs.—1 pistil, 5 stamens Seeds are not formed, the flower is sterile.

Roots:

Type of roots.—Fibrous. Roots start to grow on every part of the stem that contacts the soil, so not only at the nodes.

### Physiological and Ecological Characteristics

Good tolerance to heat and cold. Strong resistance to pests and diseases, particularly powdery mildew.

We claim:

1. A new and distinct variety of Verbena plant, substantially as herein illustrated and described, characterized particularly as to novelty by larger white flowers than other Verbena plants, which appear earlier on the plant, and a growing habit that is spreading and very vigorous.
